(19) United States
(12) Patent Application Publication
(10) Pub. No.: US 2024/0062874 A1
(43) Pub. Date: Feb. 22, 2024

(54) FOOD PROVISION SUPPORT SYSTEM FOR HEALTH MANAGEMENT, METHOD, AND RECORDING MEDIUM

(71) Applicant: NEC Corporation, Tokyo (JP)

(72) Inventors: Kazuki SUZUKI, Tokyo (JP); Youichi KATO, Tokyo (JP); Shingo NAKAI, Tokyo (JP); Naoyuki NISHIO, Tokyo (JP); Keiko TANO, Tokyo (JP); Masaki IWAMOTO, Tokyo (JP)

(73) Assignee: NEC Corporation, Tokyo (JP)

(21) Appl. No.: 18/231,058

(22) Filed: Aug. 7, 2023

(30) Foreign Application Priority Data
Aug. 17, 2022 (JP) 2022-129820

(51) Int. Cl. G16H 20/60; G16H 50/20; G16H 70/60

(52) U.S. Cl. CPC G16H 20/60; G16H 50/20; G16H 70/60; G06Q 50/12

(57) ABSTRACT

A food provision support system according to an aspect of the present disclosure sets purpose information indicating a purpose of health promotion of a subject, the purpose information including at least information on a disease that the subject is affected; acquires demand information indicating a demand related to a menu for the subject; determines, using a learning model, a menu indicating a dietary content that includes a food according to the disease indicated in the purpose information and that is relevant to the demand the learning model learned a relationship between the purpose information and the demand of the subject and the menu provided to the subject by machine learning; and requests delivery of a food relevant to the determined menu to the subject.

This application is based upon and claims the benefit of priority from Japanese Patent Application No. 2022-129820, filed on Aug. 17, 2022, the disclosure of which is incorporated herein in its entirety by reference.

### TECHNICAL FIELD

The present disclosure relates to a technique for assisting in the provision of diet.

### BACKGROUND ART

There is a technique of suggesting a dietary menu for health management. For example, a reference document (Japanese Patent Application Laid-Open No. 2008-210218) discloses that insufficient nutrients in diet of a user are specified from diet information indicating diet taken by the user, and a menu containing a large amount of the specified nutrients is suggested to the user.

The dietary content to be taken varies depending on the subject. In particular, for a subject who is concerned about a disease, it is necessary to consider the viewpoint of coping with the disease, such as prevention or treatment of the disease. That is, such a subject is generally required to have diet according to a disease, not a diet according to excess or deficiency of nutrients to be taken. It is also necessary to consider restrictions on ingredients, forms of meals, and the like depending on the subject.

### SUMMARY

An object of the present disclosure is to provide a food provision support system and the like capable of supporting the provision of diet according to a disease.

A food provision support system according to an aspect of the present disclosure includes: one or more memories storing instructions; and one or more processors configured to execute the instructions to: set purpose information indicating a purpose of health promotion of a subject, the purpose information including at least information on a disease that the subject is affected; acquire demand information indicating a demand related to a menu for the subject; determine, using a learning model, a menu indicating a dietary content that includes a food according to the disease indicated in the purpose information and that is relevant to the demand the learning model learned a relationship between the purpose information and the demand of the subject and the menu provided to the subject by machine learning; and request delivery of a food relevant to the determined menu to the subject.

In a method according to an aspect of the present disclosure, a computer executes: setting purpose information indicating a purpose of health promotion of a subject, the purpose information including at least information on a disease that the subject is affected; acquiring demand information indicating a demand related to a menu for the subject; determining, using a learning model, a menu indicating a dietary content that includes a food according to the disease indicated in the purpose information and that is relevant to the demand the learning model learned a relationship between the purpose information and the demand of the subject and the menu provided to the subject by machine learning; and requesting delivery of a food relevant to the determined menu to the subject.

A non-transitory recording medium according to one aspect of the present disclosure has stored therein a program for causing a computer to execute: setting purpose information indicating a purpose of health promotion of a subject, the purpose information including at least information on a disease that the subject is affected; acquiring demand information indicating a demand related to a menu for the subject; determining, using a learning model, a menu indicating a dietary content that includes a food according to the disease indicated in the purpose information and that is relevant to the demand the learning model learned a relationship between the purpose information and the demand of the subject and the menu provided to the subject by machine learning; and requesting delivery of a food relevant to the determined menu to the subject.

### BRIEF DESCRIPTION OF THE DRAWINGS

Exemplary features and advantages of the present disclosure will become apparent from the following detailed description when taken with the accompanying drawings in which:

FIG. 1 is a diagram schematically illustrating an example of a configuration of a food provision support system according to a first example embodiment;

FIG. 2 is a block diagram illustrating an example of a functional configuration of the food provision support system according to the first example embodiment;

FIG. 3 is a flowchart for explaining an example of an operation of the food provision support system according to the first example embodiment;

FIG. 4 is a diagram schematically illustrating an example of a configuration including a second food provision support system;

FIG. 5 is a block diagram illustrating an example of a functional configuration of a food provision support system according to a second example embodiment;

FIG. 6 is a diagram illustrating a first example of an input form according to the second example embodiment;

FIG. 7 is a diagram illustrating a second example of the input form according to the second example embodiment;

FIG. 8 is a diagram illustrating an example of confirmation information according to the second example embodiment;

FIG. 9 is a sequence diagram for explaining an example of an operation of the food provision support system according to the second example embodiment;

FIG. 10 is a block diagram illustrating an example of a functional configuration including a food provision support system according to a third example embodiment;

FIG. 11 is a diagram illustrating an example of priority information according to the third example embodiment;

FIG. 12 is a diagram illustrating an example of confirmation information according to the third example embodiment;

FIG. 13 is a sequence diagram for explaining an example of an operation of the food provision support system according to the third example embodiment;

FIG. 14 is a block diagram illustrating an example of a functional configuration including a food provision support system according to a fourth example embodiment;

FIG. 15 is a sequence diagram for explaining an example of an operation of the food provision support system according to the fourth example embodiment; and

FIG. 16 is a block diagram illustrating an example of a hardware configuration of a computer device that implements the food provision support system according to the first, second, third, and fourth example embodiments of the present disclosure.

### EXAMPLE EMBODIMENT

Next, a detailed explanation will be given for a first example embodiment with reference to the drawings.

### First Example Embodiment

An outline of a food provision support system according to a first example embodiment will be described.

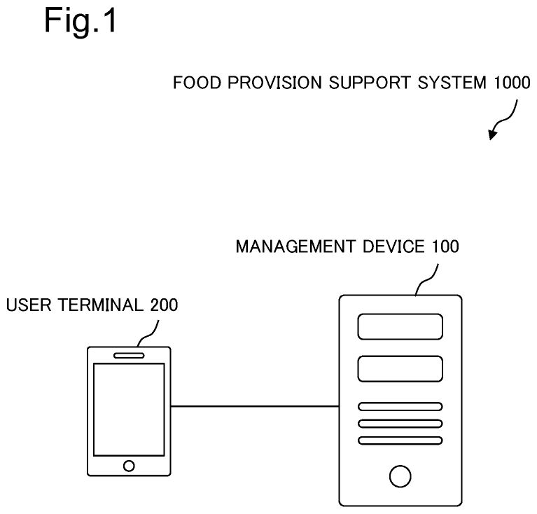

FIG. 1 is a diagram schematically illustrating an example of a configuration of a food provision support system 1000. As illustrated in FIG. 1, the food provision support system 1000 includes, for example, a management device 100 and a user terminal 200. The management device 100 and the user terminal 200 are communicably connected via a wireless or wired network.

The user terminal 200 is a terminal used by the user. For example, the user terminal 200 may be a smartphone, a tablet terminal, a personal computer, or the like. The user terminal 200 accepts an input from the user. The user uses the user terminal 200 to input, for example, information indicating the purpose of health promotion of the subject, information indicating a demand regarding a menu, and the like. Here, the user and the subject may be the same person or different persons.

The management device 100 is a computer device. The management device 100 may be, for example, a server. The management device 100 acquires information input by the user from the user terminal 200. The management device 100 determines a menu indicating dietary contents for coping with the disease of the subject based on the acquired information. Then, the management device 100 requests delivery so that the food relevant to the menu arrives at the subject.

As described above, the food provision support system 1000 is a system that supports providing the subject with a menu for coping with a disease of the subject.

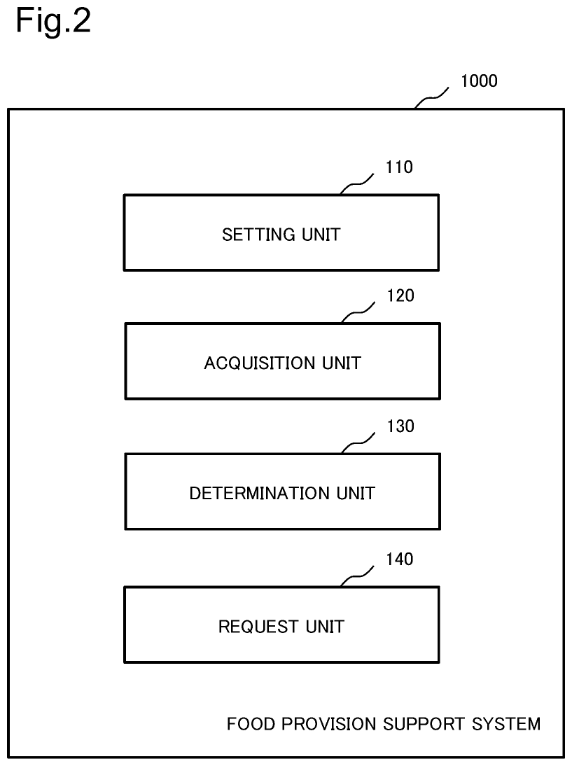

Next, an example of a functional configuration of the food provision support system 1000 will be described. FIG. 2 is a block diagram illustrating an example of a functional configuration of the food provision support system 1000. As illustrated in FIG. 2, the food provision support system 1000 includes a setting unit 110, an acquisition unit 120, a determination unit 130, and a request unit 140.

The setting unit 110 sets purpose information of the subject. The purpose information is information indicating a purpose of health promotion of the subject. For example, the purpose information includes information on a disease that the subject is likely to be affected, a disease that the subject should avoid, or a disease that the subject is affected. That is, the purpose information includes information on a disease that the subject wants to deal with for the health promotion of the subject. For example, in the user terminal 200, an input of information regarding a disease is received from the user. The setting unit 110 may set the input information regarding the disease as the purpose information.

In this manner, the setting unit 110 sets the purpose information indicating the purpose of health promotion of the subject. The setting unit 110 is an example of a setting means.

The acquisition unit 120 acquires demand information. The demand information is information indicating a demand related to a menu indicating dietary contents. That is, the demand information indicates that the subject requests the menu. For example, the demand information includes information indicating an allergy of the subject, a preference of the subject, a form of meal, a budget per meal, and the like. For example, the user terminal 200 receives an input of a demand from the user. The acquisition unit 120 may acquire the input demand as the demand information.

In this manner, the acquisition unit 120 acquires the demand information indicating the demand regarding the menu. The acquisition unit 120 is an example of an acquisition means.

The determination unit 130 determines a menu that has dietary contents according to the disease and dietary contents relevant to the demand. Specifically, the determination unit 130 determines a menu indicating dietary contents including a food according to the disease indicated by the purpose information. At this time, the determination unit may determine a menu based on food data indicating a relationship between a measure against a disease and food. The food data is, for example, a database in which the type of disease and the food are associated with each other. The food data may be a learning model in which the relationship between the dietary contents and the result of improvement in the disease is learned by machine learning.

The determination unit 130 determines a menu so as to indicate dietary contents relevant to the demand. For example, the determination unit 130 extracts a food satisfying the demand from among foods according to diseases based on the demand information. Then, the determination unit 130 determines a menu including the extracted food. The food satisfying the demand is, for example, a food excluding an ingredient that causes an allergy of the subject, and a food according to the preference of the subject.

In this manner, the determination unit 130 determines, based on the food data indicating the relationship between the measure against the disease and the food, a menu that includes dietary content including foods according to the disease indicated in the purpose information and indicates dietary contents relevant to the demand. The determination unit 130 is an example of a determination means.

The request unit 140 requests food relevant to a menu. Specifically, delivery is requested to a delivery company so that the menu determined by the determination unit 130 is provided to the subject. Here, the food may be a meal or an ingredient. That is, the delivery company delivers the meals indicated in the menu or ingredients necessary for the meals.

The request unit 140 may request delivery by transmitting information indicating the determined menu and identification information for identifying the subject to the delivery company server. In this case, the delivery company specifies the address of the delivery destination from the transmitted identification information based on, for example, contractor information in which the identification information and the address are associated with each other.

In this manner, the request unit 140 requests delivery of the food relevant to the determined menu to the subject. The request unit 140 is an example of a request means.

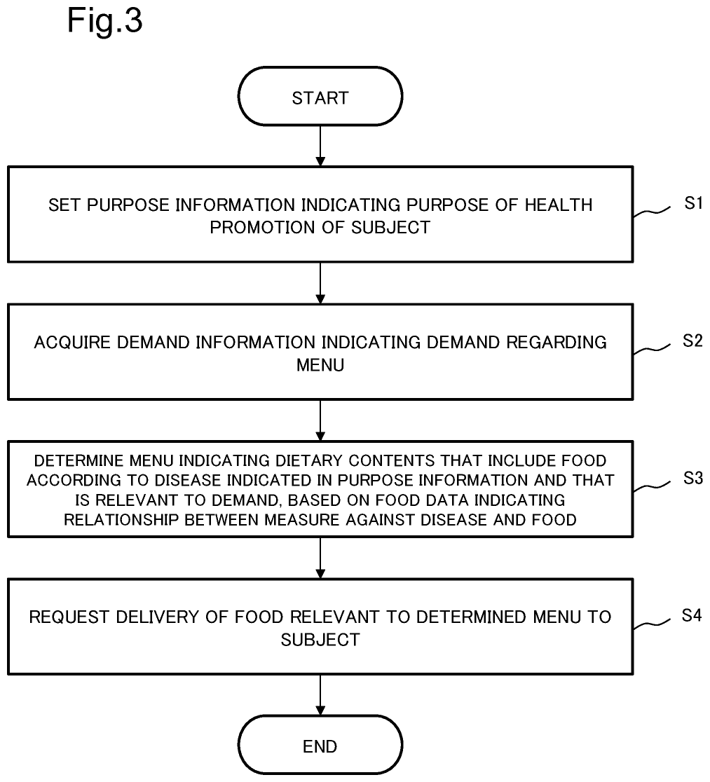

Next, an example of an operation of the food provision support system 1000 will be described with reference to FIG. 3. In the present disclosure, each step of the flowchart is expressed using a number assigned to each step, such as “S1”.

FIG. 3 is a flowchart for explaining an example of an operation of the food provision support system 1000. The setting unit 110 sets purpose information indicating a purpose of health promotion of the subject (S1). The acquisition unit 120 acquires demand information indicating a demand regarding a menu (S2). Based on the food data indicating the relationship between the measure against the disease and the food, the determination unit 130 determines a menu indicating dietary contents that include the food according to the disease indicated in the purpose information and that is relevant to the demand (S3). The request unit 140 requests delivery of the food relevant to the determined menu to the subject (S4).

As described above, the food provision support system 1000 according to the first example embodiment sets purpose information indicating a purpose of health promotion of a subject and acquires demand information indicating a demand regarding a menu. The food provision support system 1000 determines, based on food data indicating a relationship between a measure against a disease and food, a menu indicating dietary contents that include the food according to the disease indicated in the purpose information and that is relevant to the demand. Then, the food provision support system 1000 requests delivery of the food relevant to the determined menu to the subject.

As a result, the food provision support system 1000 can provide the subject with food for coping with the disease. Since the provided food is relevant to the demand, the food provision support system 1000 can provide food that is easy for the subject to eat. Furthermore, since the food is delivered to the subject, the food provision support system 1000 can suppress a burden on the subject regarding preparation of diet. That is, the food provision support system 1000 according to the first example embodiment can support the provision of diet according to a disease.

### Second Example Embodiment

Next, a food provision support system according to a second example embodiment will be described. In the second example embodiment, a further example of the food provision support system described in the first example embodiment will be described. The description of contents overlapping with the first example embodiment will be partially omitted.

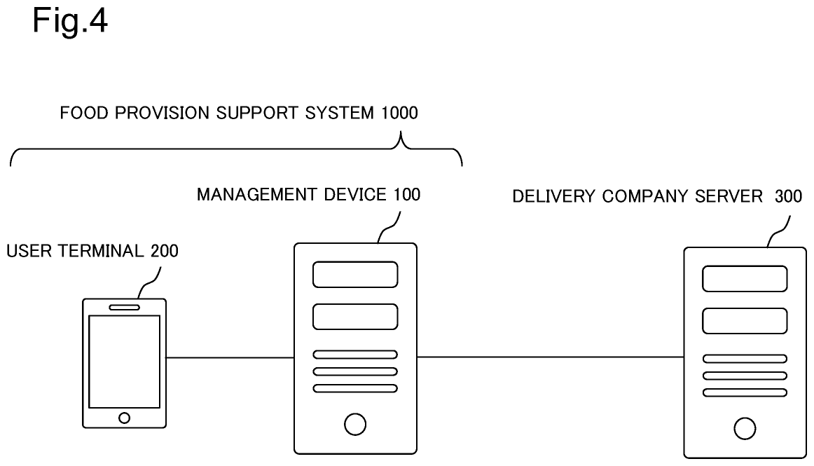

FIG. 4 is a diagram schematically illustrating an example of a configuration including the food provision support system 1000. As illustrated in FIG. 4, the food provision support system 1000 includes a management device 100 and a user terminal 200. The management device 100 is communicably connected to a delivery company server 300 via a wired or wireless network.

[Details of Food Provision Support System 1000]

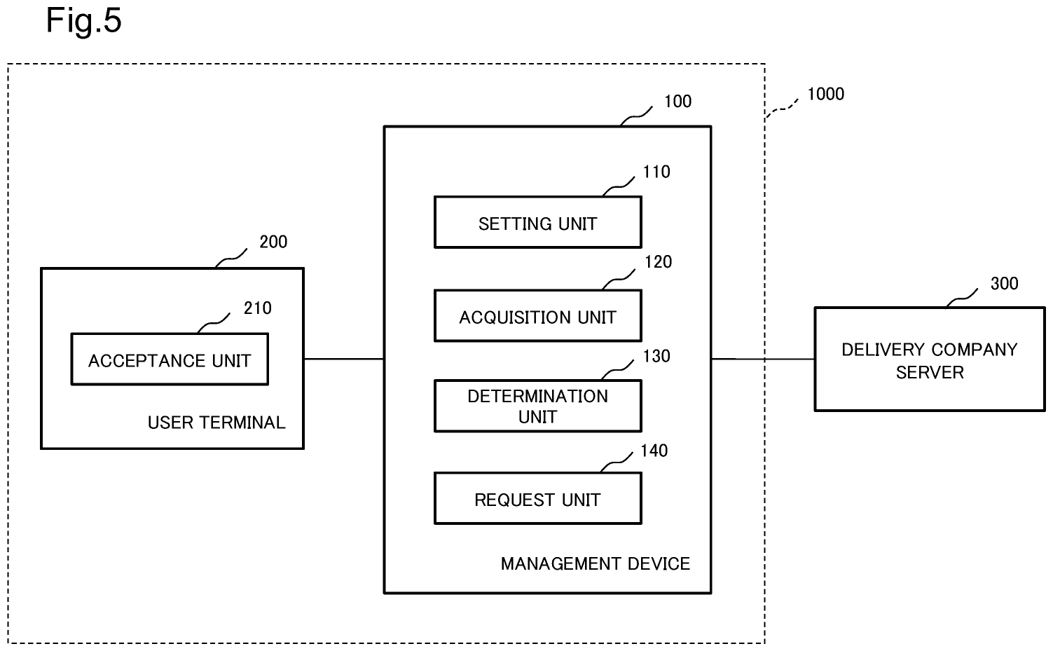

FIG. 5 is a block diagram illustrating an example of a functional configuration of the food provision support system 1000. As illustrated in FIG. 5, the management device 100 includes a setting unit 110, an acquisition unit 120, a determination unit 130, and a request unit 140. The user terminal 200 includes an acceptance unit 210.

The acceptance unit 210 accepts an input from a user who uses the user terminal 200. The user may be the same person as the subject or may be a person different from the subject. For example, the user may be a relative such as a guardian of the subject, or may be a medical worker such as an attending doctor of the subject.

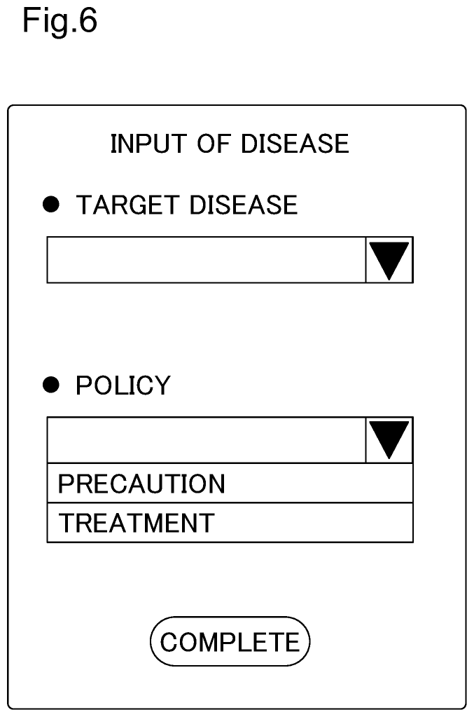

The acceptance unit 210 accepts an input of information regarding a disease of the subject from the user. FIG. 6 is a diagram illustrating a first example of the input form. More specifically, FIG. 6 is a diagram illustrating an example of an input form for accepting an input of information regarding a disease of a subject. For example, a display control unit of the management device 100 (not illustrated) may display the input form on the user terminal 200. The acceptance unit 210 accepts an input of information regarding a disease through an input form as illustrated in FIG. 6, for example. In the example of FIG. 6, “target disease” and “policy” are input. For example, types of diseases such as diabetes, kidney disease, and hypertension are input in the column of “target disease”. The type of disease is not limited to this example. In the present disclosure, the type of disease includes information such as advanced age and pregnancy. That is, the type of disease does not necessarily indicate the type of disease. In the column of “policy”, a policy of coping with a target disease is input. The coping policy is prevention, treatment, and the like.

In this manner, the acceptance unit 210 accepts the input of the information regarding the disease of the subject from the user. The information regarding the disease may include information indicating a policy of coping with the disease in addition to the information indicating the disease. The acceptance unit 210 transmits the input information to the management device 100. The setting unit 110 sets the transmitted information regarding the disease as the purpose information of the subject.

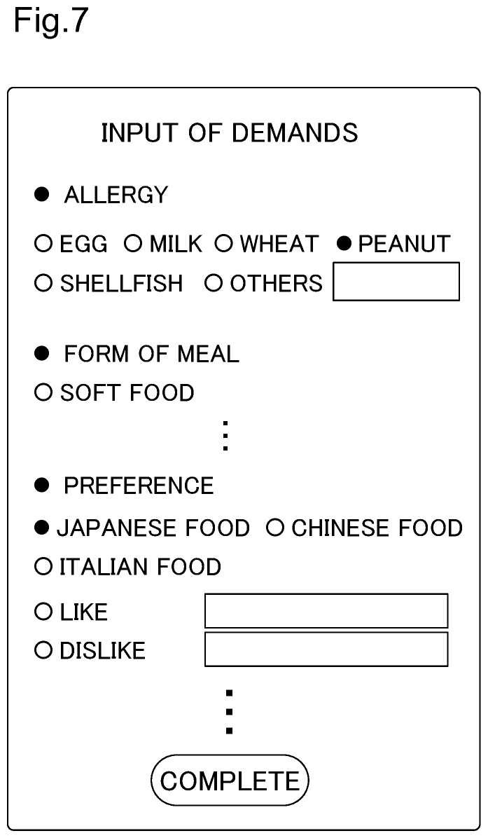

The acceptance unit 210 accepts an input of a demand from the user. FIG. 7 is a diagram illustrating a second example of the input form. More specifically, FIG. 7 is a diagram illustrating an example of an input form for accepting a demand input. For example, a display control unit of the management device 100 (not illustrated) may display the input form on the user terminal 200. The acceptance unit 210 accepts an input of a demand through an input form as illustrated in FIG. 7, for example. In the example of FIG. 7, “allergy”, “form of meal”, and “preference” are input. In the “allergy” column, ingredients that cause an allergic reaction of the subject, such as eggs and milk, are input. In the “form of meal”, the hardness of the meal, the size of ingredients, and the like are input. The information on the form of the meal is related to, for example, the degree to which the subject can masticate. In the “preference”, a genre of food such as Japanese food or Chinese food, and the like of the subject are input. The present disclosure is not limited to this example, and the demand may include a budget for the menu.

In this manner, the acceptance unit 210 accepts a demand input from the user. The demand includes information indicating at least one of a form of a meal, an allergy of the subject, a preference of the subject, and a budget for a menu. The acceptance unit 210 transmits information indicating the input demand to the management device 100. The acquisition unit 120 acquires information indicating the transmitted demand as the demand information of the subject.

The determination unit 130 determines a menu based on the set purpose information and the acquired demand information. At this time, the determination unit 130 determines a menu using food data indicating a relationship between a measure against a disease and food.

The food data is, for example, a database in which the type of disease and the food are associated with each other. The database may be information in which ingredients or meals containing nutrients necessary for coping with a disease are associated with each disease. Not limited to this example, the database may be a collection of dietary contents of a person whose disease has been improved for each disease. In a case where the food data is such a database, the determination unit 130 extracts food according to the disease indicated in the purpose information.

The food data may be a database in which a policy of coping with a disease and food are associated with each other. In this case, the determination unit 130 extracts a food according to a policy of coping with the disease indicated in the purpose information.

The food data may be a learning model in which the relationship between the dietary contents and the result of improvement in the disease is learned by machine learning. For example, the learning model is a model in which a relationship between food taken in by a person suffering from a specific disease and whether the disease of the person is improved is machine-learned. In this case, for example, the determination unit 130 inputs information indicating the disease indicated in the purpose information to the learning model. Then, information indicating a food having a tendency that the disease is improved is output by the learning model. The determination unit 130 may extract the information indicating the food output by the learning model as the food according to the disease of the subject. The learning model is learned by, for example, deep learning using a neural network, but is not limited to this example. The learning model may be generated by the determination unit 130.

Then, the determination unit 130 determines a menu based on the demand information. Specifically, the determination unit 130 determines a menu including foods that satisfy the demand indicated in the demand information among foods extracted based on the food data. For example, if the demand information includes information indicating an allergy of the subject, a menu excluding the ingredient that causes the allergy is determined. For example, when the demand information includes information regarding preference, a menu including food according to the preference is determined. Specifically, in a case where “Japanese food” has been selected as the information regarding the preference, the determination unit 130 determines a Japanese menu. When determining a menu, the determination unit 130 may refer to meal information in which a meal, an ingredient used for the meal, a cost for the meal, and a genre of the meal are associated with each other. For example, the determination unit 130 may determine a menu by selecting a meal that satisfies the demand indicated in the demand information from the meal information. The meal information may be stored in a storage device (not illustrated) included in the management device 100, or may be stored in an external device (not illustrated) communicably connected to the management device 100.

In a case where a food according to a policy of coping with a disease is extracted, the determination unit 130 determines a menu indicating dietary contents including a food according to the policy.

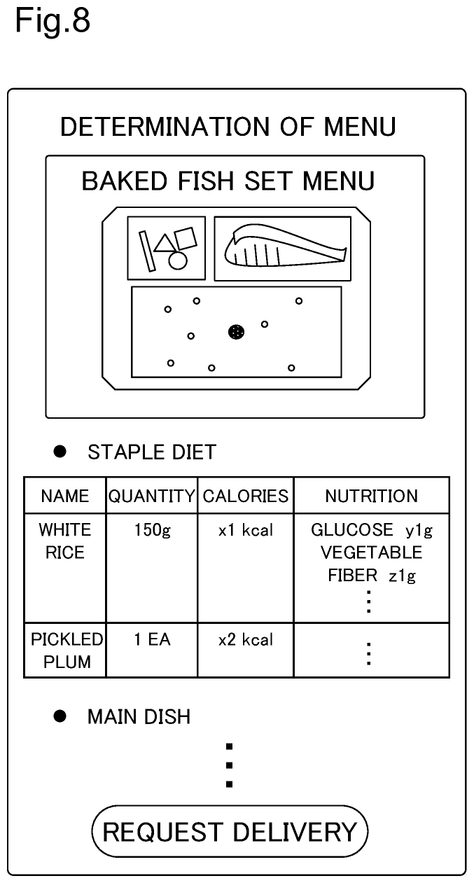

The request unit 140 requests delivery of the determined menu. At this time, the request unit 140 may prompt the user to confirm the determined menu. For example, the request unit 140 causes the user terminal 200 to display confirmation information for causing the user to confirm a menu. FIG. 8 is information indicating an example of the confirmation information. As illustrated in FIG. 8, the confirmation information includes information indicating the determined menu and information regarding ingredients included in the menu. In the example of FIG. 8, the information regarding the ingredient indicates the name, quantity, calories, and nutrients of the ingredient. For example, the user visually recognizes the confirmation information displayed on the user terminal 200. Then, in a case where the user agrees with the menu indicated in the confirmation information, the user performs an input for requesting delivery. In this way, the request unit 140 can support the user's decision-making regarding menu determination. In the example of FIG. 8, when the button describing “request delivery” is selected by the user, the request unit 140 requests the delivery company to deliver the menu. The delivery company includes a company that performs delivery, a company that prepares food relevant to a menu, and the like.

For example, the request unit 140 requests the delivery of the meal indicated in the menu or the ingredient used for the meal by transmitting the information indicating the determined menu and the identification information of the user to the delivery company server 300. The delivery company server 300 specifies the address of the delivery destination based on the identification information. For example, the contractor information in which the identification information and the address are associated with each other is stored in the delivery company server 300. The delivery company server 300 specifies the address relevant to the transmitted identification information as the address of the delivery destination based on the contractor information.

[Operation Example of Food Provision Support System 1000]

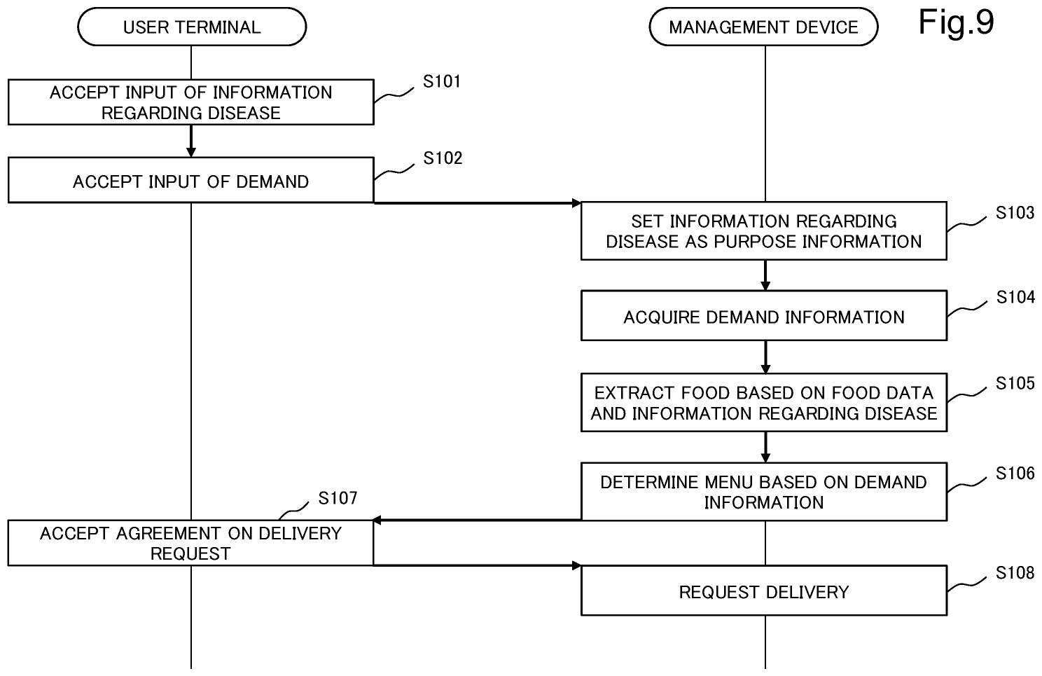

Next, an example of an operation of the food provision support system 1000 will be described with reference to FIG. 9.

FIG. 9 is a sequence diagram illustrating an example of the operation of the food provision support system 1000. First, an input form is displayed on the user terminal 200. The acceptance unit 210 accepts an input of information regarding a disease (S101). The acceptance unit 210 accepts an input of a demand (S102). Then, the acceptance unit 210 transmits the received information on the disease and the demand to the management device 100.

The setting unit 110 sets the transmitted information regarding the disease as the purpose information (S103). The acquisition unit 120 acquires the transmitted demand as the demand information (S104). The determination unit 130 extracts a food according to a disease based on the food data and the information regarding the disease (S105). The determination unit 130 determines a menu using the extracted food based on the demand information (S106).

Then, confirmation information for accepting agreement on the delivery request is displayed on the user terminal 200. When the acceptance unit 210 accepts the agreement on the delivery request (S107), the request unit 140 requests the delivery (S108).

As described above, the food provision support system 1000 according to the second example embodiment sets purpose information indicating a purpose of health promotion of a subject and acquires demand information indicating a demand regarding a menu. The food provision support system 1000 determines, based on food data indicating a relationship between a measure against a disease and food, a menu indicating dietary contents that include the food according to the disease indicated in the purpose information and that is relevant to the demand. Then, the food provision support system 1000 requests delivery of the food relevant to the determined menu to the subject.

As a result, the food provision support system 1000 can provide the subject with food for coping with the disease. Since the provided food is relevant to the demand, the food provision support system 1000 can provide food that is easy for the subject to eat. Furthermore, since the food is delivered to the subject, the food provision support system 1000 can suppress a burden on the subject regarding preparation of diet. That is, the food provision support system 1000 according to the second example embodiment can support the provision of diet according to a disease.

The purpose information may include information indicating a policy of coping with the disease of the subject. Then, the food provision support system 1000 may determine a menu indicating dietary contents including foods according to the policy. Even in the same disease, the correspondence may change between prevention and treatment. On the other hand, since the food provision support system 1000 can determine a menu according to a policy of coping with a disease, it is possible to support the provision of diet more suitable for the subject.

The demand information may include information indicating a demand regarding at least one of a form of meal, an allergy of the subject, a preference of the subject, and a budget for a menu. As a result, the food provision support system 1000 can determine a menu relevant to a disease in consideration of a form of meal demanded by the subject, allergy, preference, and budget.

[Modification 1]

In the above-described example embodiment, an example in which the food provision support system 1000 includes the user terminal 200 and the management device 100 has been described. On the other hand, the configuration of the food provision support system 1000 is not limited to this example. For example, the food provision support system 1000 may further include a delivery company server 300.

The food provision support system 1000 may be implemented in the user terminal 200. That is, the user terminal 200 may include the setting unit 110, the acquisition unit 120, the determination unit 130, and the request unit 140. In this case, the user terminal 200 is communicably connected to the delivery company server 300.

[Modification 2]

The information about the disease may be input by a medical worker. That is, the management device 100 may acquire information regarding the disease from the terminal used by the medical worker and set the acquired information as the purpose information.

The subject or the guardian of the subject may not be familiar with the disease. On the other hand, the food provision support system 1000 can set more accurate purpose information by receiving input of information regarding a disease from a medical worker such as an attending doctor of the subject.

[Modification 3]

In the above-described example embodiment, an example has been described in which a food is extracted from food data and a menu is determined from the extracted food. The menu determination method is not limited to this example.

For example, the determination unit 130 may determine a menu by using a learning model in which the purpose information and the demand are input and the menu is output. For example, the learning model is a model in which a relationship between the purpose information and the demand of the subject and the menu provided to the subject is machine-learned. The learning model is learned by, for example, deep learning using a neural network, but is not limited to this example.

The learning model used for the menu determination may be generated by the determination unit 130. Then, the learning model used for the menu determination may be retraining. For example, when the user visually recognizes the confirmation information displayed on the user terminal 200 and the user disagrees with the menu indicated in the confirmation information, the user will adjust the menu. The request unit 140 accepts the user's modification content. The determination unit 130 receives the modification content from the request unit 140 and uses it for retraining the learning model along with the purpose information and the demand of the subject.

### Third Example Embodiment

Next, a food provision support system according to a third example embodiment will be described. In the third example embodiment, a further example of determining a menu in consideration of the demand information will be described. A part of the description overlapping with the first example embodiment and the second example embodiment will be omitted.

The subject should be provided with a menu for coping with the disease. Therefore, in some cases, a menu that satisfies all the demands included in the demand information cannot be configured. Therefore, in the third example embodiment, an example in which priority is set to the demand information will be described.

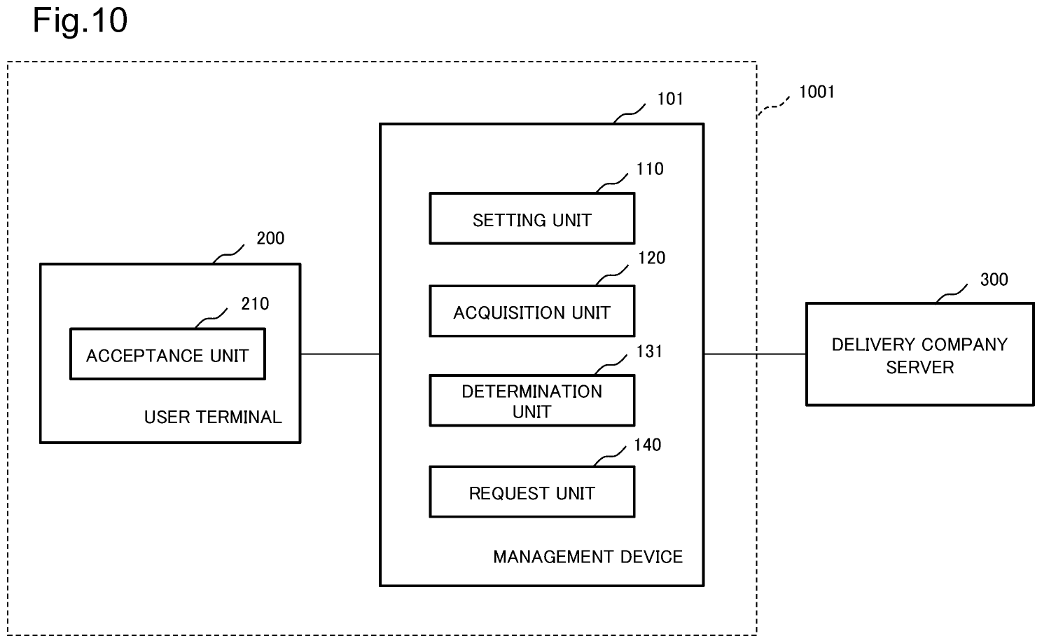

FIG. 10 is a block diagram illustrating an example of a functional configuration including a food provision support system 1001. FIG. 10 is an example in which a food provision support system 1001 is illustrated instead of the food provision support system 1000 in the configuration example of FIG. 4. The food provision support system 1001 includes a management device 101 and a user terminal 200. The management device 101 is communicably connected to the delivery company server 300.

The management device 101 includes a setting unit 110, an acquisition unit 120, a determination unit 131, and a request unit 140.

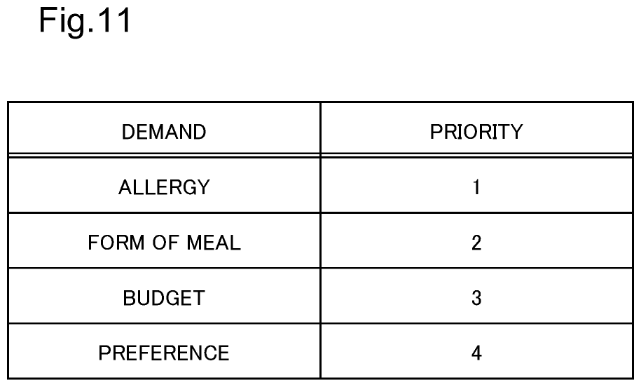

The determination unit 131 determines a menu according to the demand information and the priority relevant to various demands. The priority is set in advance for the demand. For example, priority information in which a demand and a priority are associated with each other is generated. FIG. 11 is a diagram illustrating an example of the priority information. For example, a priority “1” is associated with “allergy”, and a priority “4” is associated with “preference”. In this example, it is assumed that the priority becomes higher as the value of the priority becomes closer to 1. For example, when the subject has an allergy, it is essential to exclude allergic ingredients from the menu. That is, the demand related to allergy is set to have a high priority. On the other hand, the demand regarding the preference of the subject is not essential as compared with the demand regarding the allergy. Therefore, the demand regarding the preference of the subject is set to have a low priority. The determination unit 131 determines a menu that satisfies a demand with a high priority according to the priority. In other words, the determination unit 131 determines a menu that at least satisfies a demand for which a priority equal to or higher than a predetermined threshold is set.

For example, the determination unit 131 determines a menu from foods extracted from the food data and the purpose information based on the priority information. At this time, when the menu that satisfies all the demands cannot be configured, the determination unit 131 excludes the demand with low priority and configures the menu again. In the example of FIG. 11, the menu is configured excluding the demand regarding the preference of the subject. In a case where the menu cannot be configured yet, the menu may be configured again by excluding a demand with the next lowest priority. In the example of FIG. 11, the menu is configured excluding the demand regarding the preference of the subject and the demand regarding the budget.

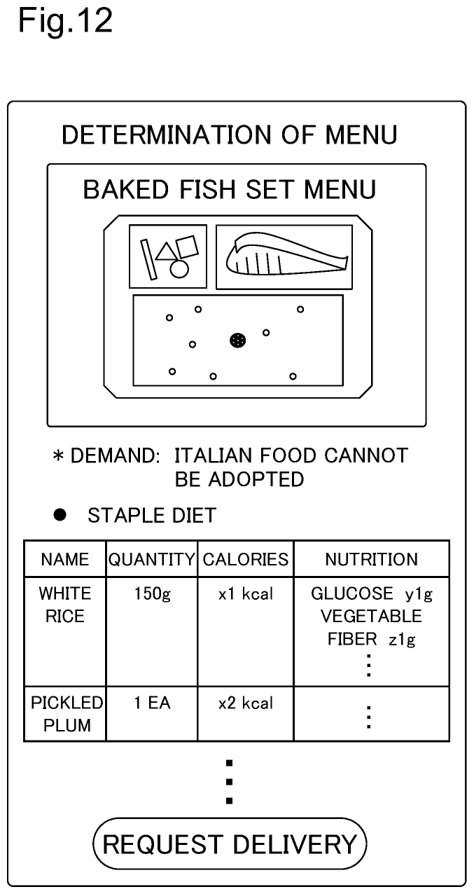

Then, when the determination unit 131 determines a menu, the request unit 140 causes the user terminal 200 to display confirmation information. FIG. 12 is a diagram illustrating an example of the confirmation information. For example, it is assumed that “Italian” is selected as the demand regarding the preference of the subject. Then, in a case where the demand regarding the preference has not been considered, information indicating that the demand has not been considered is included in the confirmation information. In the example of FIG. 12, a sentence “Demand: Italian cannot be adopted” is displayed.

[Operation Example of Food Provision Support System 1001]

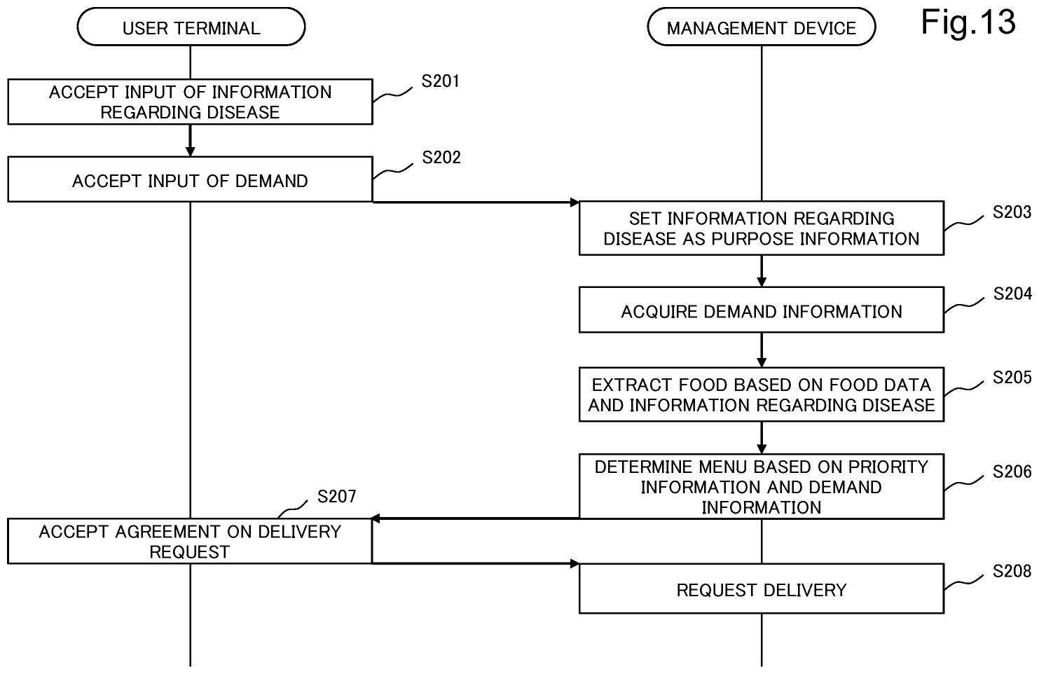

Next, an example of an operation of the food provision support system 1001 will be described with reference to FIG. 13.

FIG. 13 is a sequence diagram illustrating an example of the operation of the food provision support system 1001. The description of steps similar to those in the sequence diagram of FIG. 9 is omitted.

Since the processing of S201 to S205 is similar to the processing of S101 to S105 of FIG. 9, description of the processing is omitted.

The determination unit 131 determines a menu based on the priority information and the demand information (S206). Specifically, the determination unit 131 first configures a menu that satisfies all the demands. In a case where the menu cannot be configured, the determination unit 131 reconfigures the menu excluding the demand with low priority. In a case where a menu that satisfies a demand other than the excluded demand can be configured, the determination unit 131 determines a menu.

In a case where there is a demand that has not been satisfied, confirmation information including information indicating that the demand has not been satisfied is displayed on the user terminal 200. Then, the processing after S207 is performed. The processing of S207 and S208 is similar to the processing of S107 and S108 in FIG. 9.

As described above, in the third example embodiment, the priority may be set to the demand indicated in the demand information. Then, the food provision support system 1001 may determine a menu that at least satisfies a demand for which a priority equal to or higher than a predetermined threshold is set. The subject should be provided with a menu to address the disease. On the other hand, when it is attempted to satisfy all the demands of the subject, a menu for coping with a disease may not be configured. On the other hand, the food provision support system 1001 can propose a menu that satisfies at least essential demands even when all the demands cannot be satisfied.

### Fourth Example Embodiment

Next, a food provision support system according to a fourth example embodiment will be described. In the fourth example embodiment, a further example of a case where ingredients constituting a menu are delivered will be described. A part of description overlapping with the first example embodiment, the second example embodiment, and the third example embodiment will be omitted.

In a case where an ingredient is delivered to the subject, there is a possibility that the subject possesses a part of the ingredient included in the menu. Therefore, in the fourth example embodiment, an example of requesting the delivery of the ingredient in consideration of the ingredient owned by the subject will be described.

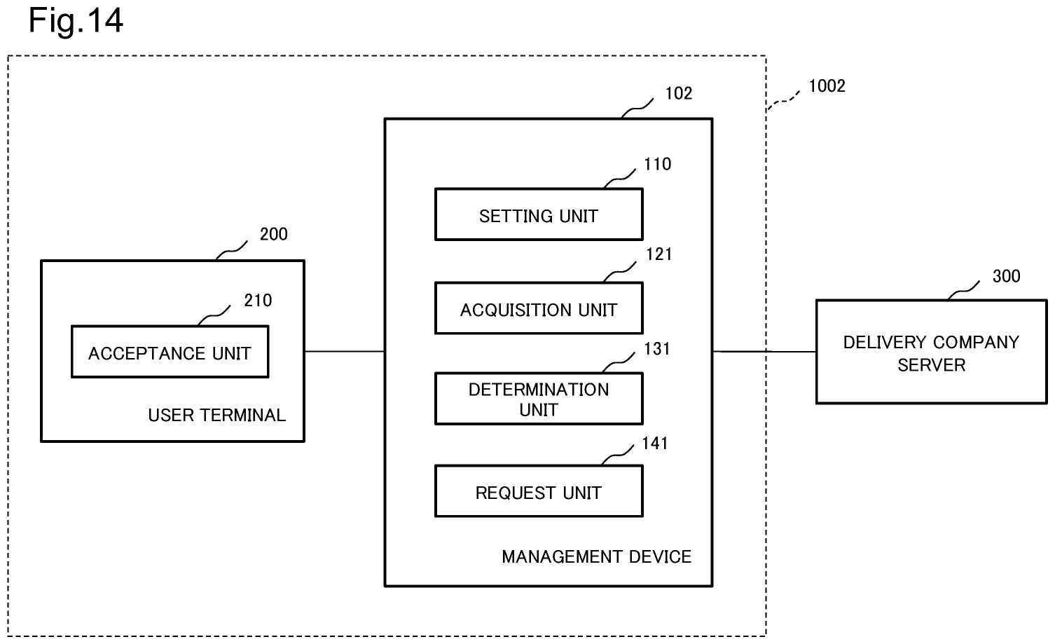

FIG. 14 is a block diagram illustrating an example of a functional configuration including a food provision support system 1002. FIG. 14 is an example in which the food provision support system 1002 is illustrated instead of the food provision support system 1000 in the configuration example of FIG. 4. The food provision support system 1002 includes a management device 102 and a user terminal 200. The management device 102 is communicably connected to the delivery company server 300.

The management device 102 includes a setting unit 110, an acquisition unit 121, a determination unit 131, and a request unit 141.

The acquisition unit 121 acquires the held ingredient information. The held ingredient information is information regarding an ingredient held by the subject. The held ingredient information is not limited to this example, and may be information regarding an ingredient possessed by the user of the user terminal 200 such as a guardian of the subject. For example, the acceptance unit 210 accepts an input of the held ingredient from the user. Then, the acceptance unit 210 transmits the input information to the management device 102. The acquisition unit 121 acquires the transmitted information regarding the ingredient as the held ingredient information. In this manner, the acquisition unit 121 acquires the held ingredient information that is information regarding an ingredient held by the subject.

The held ingredient information may be obtained, for example, based on images. For instance, in an image processing server (not shown), type and quantity of ingredients are detected using Convolutional Neural Network (CNN) based object detection technology, based on the images obtained from cameras attached to household appliances such as a refrigerator of the subject. In this case, for example, the acceptance unit 210 accepts instructions for ingredient recognition from the user and requests the held ingredient information from the image processing server. The image processing server, triggered by the request from the acceptance unit 210, acquires the camera images and detects the type and the quantity of ingredients based on the camera images. The image processing server then sends this information as the held ingredient information to the acceptance unit 210. The acceptance unit 210 sends the received held ingredient information to the management device 102. Before sending, the acceptance unit 210 may present the held ingredient information to the user for confirmation.

The request unit 141 requests the delivery of the ingredient based on the held ingredient information. Specifically, the request unit 141 extracts ingredients not included in the held ingredient information among the ingredients constituting the determined menu. In other words, the request unit 141 compares the held ingredient information with an ingredient to be used for the meal indicated in the menu, and extracts an insufficient ingredient among the ingredients to be used for the meal. Then, the request unit 141 requests delivery of the extracted ingredient. Specifically, the request unit 141 transmits identification information of the subject and information indicating the extracted ingredients to the delivery company server 300. In this manner, the request unit 141 requests delivery of an insufficient ingredient among ingredients to be used for the meal indicated in the menu based on the held ingredient information.

[Operation Example of Food Provision Support System 1002]

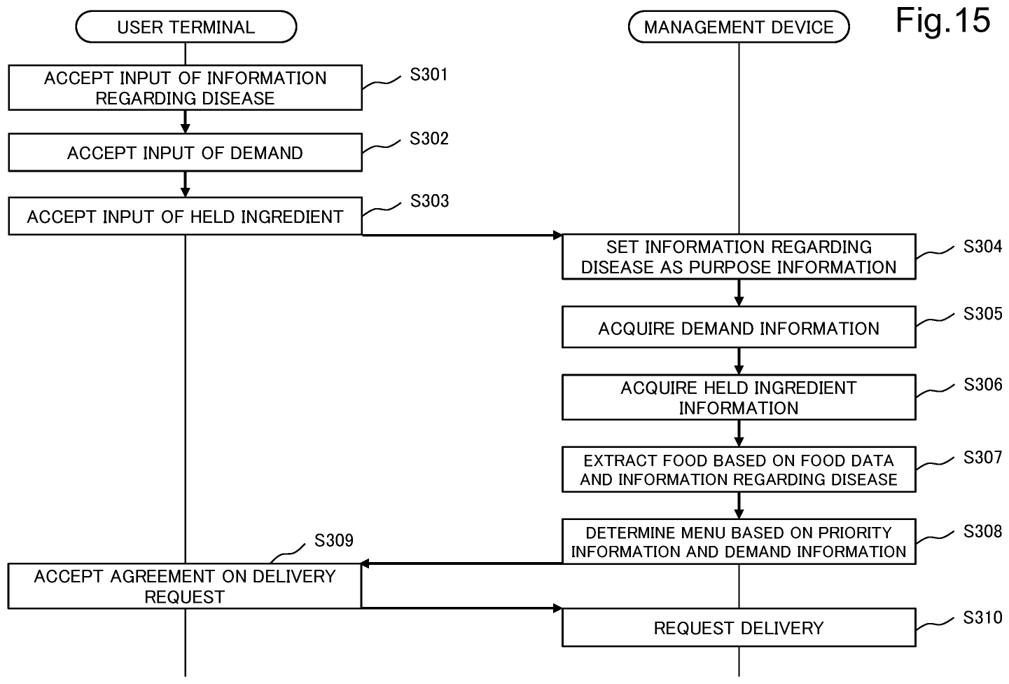

Next, an example of an operation of a food provision support system 1003 will be described with reference to FIG. 15.

FIG. 15 is a sequence diagram illustrating an example of the operation of the food provision support system 1003. The description of steps similar to those in the sequence diagram of FIG. 13 is omitted.

Since the processing of S301 and S302 is similar to the processing of S201 and S202 of FIG. 13, description of the processing is omitted.

The acceptance unit 210 accepts an input of the held ingredient (S303). Then, the acceptance unit 210 transmits the input information regarding the ingredient to the management device 102.

The processing of S304 and S305 is similar to the processing of S203 and S204 of FIG. 13. The acquisition unit 121 acquires the transmitted information regarding the ingredient as the held ingredient information (S306).

Then, the processing of S307 to S309 is performed. The processing of S307 to S309 is similar to the processing of S205 to S207 of FIG. 13. Thereafter, the request unit 141 requests delivery (S310). At this time, the request unit 141 requests delivery of an insufficient ingredient among ingredients to be used for the meal indicated in the determined menu.

As described above, in a case where the delivery of the ingredient is requested, the food provision support system 1002 according to the fourth example embodiment acquires the held ingredient information that is information regarding the ingredient held by the subject, and requests the delivery of an insufficient ingredient among the ingredients used for the meal indicated in the menu based on the held ingredient information. As a result, the food provision support system 1002 can prevent an ingredient owned by the subject from being delivered. Therefore, the food provision support system 1002 can prevent discarding of ingredients and reduce the cost for preparing ingredients for the subject.

The dietary content to be taken varies depending on the subject. However, it may be difficult for the subject to prepare diet according to such a disease. According to the present disclosure, it is possible to support the provision of diet according to a disease.

<Configuration Example of Hardware of Food Provision Support System>

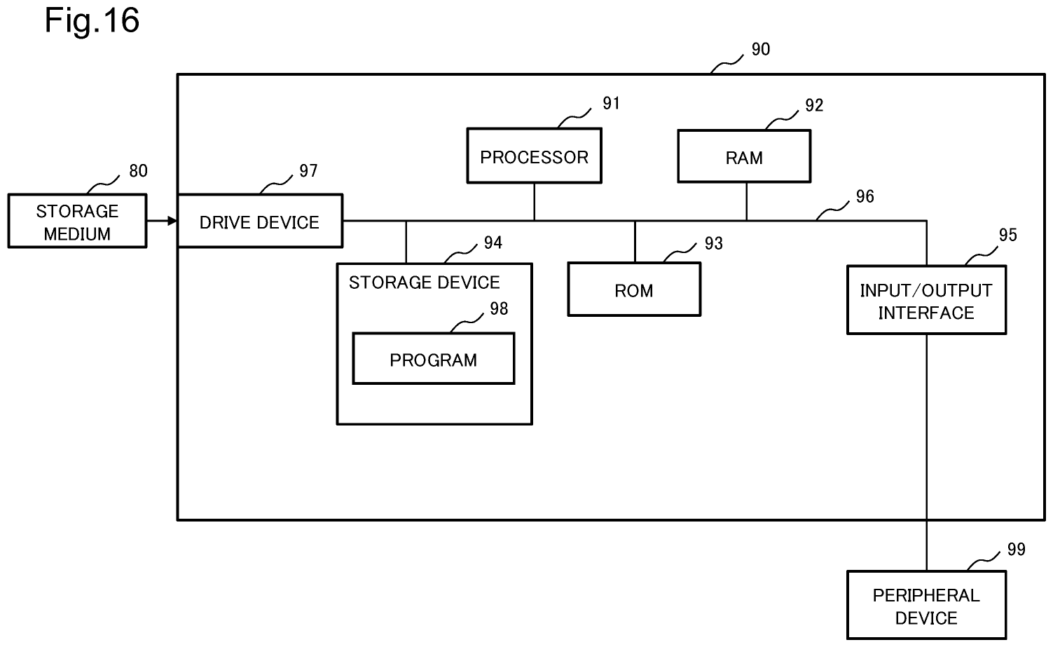

Hardware constituting the above-described first, second, third, and fourth example embodiments of the food provision support system will be described. FIG. 16 is a block diagram illustrating an example of a hardware configuration of a computer device that implements the food provision support system according to each example embodiment. In a computer device 90, the food provision support system and the dietary life management method described in each example embodiment and each modification are achieved. For example, each of the management device and the user terminal described in each example embodiment and each modification may have the hardware configuration illustrated in FIG. 16. In a case where the management device and the user terminal are integrated, the integrated device may have the hardware configuration illustrated in FIG. 16.

As illustrated in FIG. 16, the computer device 90 includes a processor 91, a random access memory (RAM) 92, a read only memory (ROM) 93, a storage device 94, an input/output interface 95, a bus 96, and a drive device 97. The food provision support system may be implemented by a plurality of electric circuits.

The storage device 94 stores a program (computer program) 98. The processor 91 executes the program 98 of the present food provision support system using the RAM 92. Specifically, for example, the program 98 includes a program that causes a computer to execute the processing of the signal processing device described in FIGS. 3, 9, 13, and 15. The processor 91 executes the program 98 to implement the functions of the components of the present food provision support system. The program 98 may be stored in the ROM 93. The program 98 may be recorded in the storage medium 80 and read using the drive device 97, or may be transmitted from an external device (not illustrated) to the computer device 90 via a network (not illustrated).

The input/output interface 95 exchanges data with a peripheral device (keyboard, mouse, display, etc.) 99. The input/output interface 95 functions as a means for acquiring or outputting data. The bus 96 connects the components

There are various modifications of the method for implementing the food provision support system. For example, the food provision support system can be achieved as a dedicated device. The food provision support system can be implemented based on a combination of a plurality of devices.

Processing methods for causing a storage medium to record a program for implementing configurations in a function of each example embodiment, reading the program recorded in the storage medium as a code, and executing the program in a computer are also included in the scope of each example embodiment. That is, a computer-readable storage medium is also included in the scope of each example embodiment. A storage medium in which the above-described program is recorded and the program itself are also included in each example embodiment.

The storage medium is, for example, a floppy (registered trademark) disk, a hard disk, an optical disk, a magneto-optical disk, a compact disc (CD)-ROM, a magnetic tape, a nonvolatile memory card, or a ROM, but is not limited to this example. The program recorded in the storage medium is not limited to a program that executes processing alone, and programs that operate on an operating system (OS) to execute processing in cooperation with other software and functions of an extension board are also included in the scope of each example embodiment.

While present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims.

The above-described example embodiments and modifications can be appropriately combined.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed disclosure even if the claims are amended during prosecution.

1. A food provision support system comprising:
one or more memories storing instructions; and
one or more processors configured to execute the instructions to:
set purpose information indicating a purpose of health promotion of a subject, the purpose information including at least information on a disease that the subject is affected;
acquire demand information indicating a demand related to a menu for the subject;
determine, using a learning model, a menu indicating a dietary content that includes a food according to the disease indicated in the purpose information and that is relevant to the demand the learning model learned a relationship between the purpose information and the demand of the subject and the menu provided to the subject by machine learning; and
request delivery of a food relevant to the determined menu to the subject.

2. The food provision support system according to claim 1, wherein
the purpose information includes information indicating a policy of coping with a disease of the subject, and
the one or more processors are configured to execute an instruction to determine a menu indicating a dietary content including a food according to the policy.

3. The food provision support system according to claim 1, wherein
the demand information includes information indicating a demand regarding at least one of a form of a meal, an allergy of the subject, a preference of the subject, and a budget for a menu.

4. The food provision support system according to claim 3, wherein
a priority is set to the demand indicated in the demand information, and
the one or more processors are configured to execute an instruction to determine a menu that at least satisfies a demand set with a priority equal to or higher than a predetermined threshold.

5. The food provision support system according to claim 1, wherein
the one or more processors are configured to execute an instruction to request delivery of a meal indicated in the menu or an ingredient used for the meal, by transmitting information indicating the determined menu and identification information of the subject to a delivery company server.

6. The food provision support system according to claim 5, wherein
the one or more processors are configured to execute, when delivery of an ingredient is requested, the instructions to:
acquire held ingredient information that is information regarding an ingredient held by the subject; and
request delivery of an insufficient ingredient among ingredients to be used for a meal indicated in the menu based on the held ingredient information.

7. The food provision support system according to claim 1, wherein
the food data is a database in which types of diseases and foods are associated with each other.

8. The food provision support system according to claim 1, wherein
the one or more processors are configured to execute an instruction to provide confirmation information to user for decision making before request delivery of a food, the confirmation information including information indicating the determined menu and information regarding ingredients included in the menu.

9. A method for causing a computer to execute:
setting purpose information indicating a purpose of health promotion of a subject, the purpose information including at least information on a disease that the subject is affected;
acquiring demand information indicating a demand related to a menu for the subject;
determining, using a learning model, a menu indicating a dietary content that includes a food according to the disease indicated in the purpose information and that is relevant to the demand the learning model learned a relationship between the purpose information and the demand of the subject and the menu provided to the subject by machine learning; and
requesting delivery of a food relevant to the determined menu to the subject.

10. A non-transitory recording medium having stored therein a program for causing a computer to execute:
setting purpose information indicating a purpose of health promotion of a subject, the purpose information including at least information on a disease that the subject is affected;
acquiring demand information indicating a demand related to a menu for the subject;
determining, using a learning model, a menu indicating a dietary content that includes a food according to the disease indicated in the purpose information and that is relevant to the demand the learning model learned a relationship between the purpose information and the demand of the subject and the menu provided to the subject by machine learning; and
requesting delivery of a food relevant to the determined menu to the subject.
